(19) United States
(12) Patent Application Publication
(10) Pub. No.: US 2014/0338670 A1
(43) Pub. Date: Nov. 20, 2014

(54) Positive Pressure Adapter

(71) Applicant: Draeger Safety, Inc., Pittsburgh, PA (US)

(72) Inventors: Eric J. Robey, Colliers, WV (US); William E. Dedig, Jr., Pittsburgh, PA (US); Robert H. Sell, Oakdale, PA (US)

(73) Assignee: DRAEGER SAFETY, INC., Pittsburgh, PA (US)

(21) Appl. No.: 13/895,918

(22) Filed: May 16, 2013

(51) Int. Cl. A61M 16/08; A61M 16/20; A61M 16/06

(52) U.S. Cl. CPC A61M 16/0833; A61M 16/06; A61M 16/20
USPC 128/205.24

(57) ABSTRACT

Described herein is an adapter having a housing defining an internal passageway, a first connecting end portion having a first opening to the internal passageway, a second connecting end portion having a second opening into the internal passageway, and an exhalation valve disposed on the housing. The first connecting end portion is configured to mechanically and fluidically couple with a lung demand valve. The second connecting end portion is configured to mechanically and fluidically couple with a mask of a closed circuit breathing apparatus. Related apparatus, systems, and/or methods of use are also described.

### TECHNICAL FIELD

The subject matter described herein relates to an adapter for interchangeable use with disparate breathing apparatus components.

### BACKGROUND

A closed circuit breathing apparatus (CCBA) or “re-breather” is a portable device that provides breathable oxygen while isolating the wearer's respiratory tract from contaminated or immediately dangerous to life or health (IDLH) environment. The CCBA is typically worn by a worker and provides respiratory protection with an oxygen source during the worker's tasks. The CCBA provides protection for a limited period of time, such as up to 4 hours. In an emergency situation, as the respirable gas of the CCBA is depleted, the CCBA can be exchanged for another system such as a SCBA (Self-Contained Breathing Apparatus) or a Rapid Intervention Team (RIT) Pack to receive a fresh supply of respirable gas from the second system.

### SUMMARY

In one aspect, an adapter device is disclosed. The adapter includes a housing defining an internal passageway. The adapter includes a first connecting end portion of the housing having a first opening to the internal passageway. The first connecting end portion is configured to mechanically and fluidically couple with a lung demand valve. The adapter includes a second connecting end portion of the housing having a second opening into the internal passageway. The second connecting end portion is configured to mechanically and fluidically couple with a mask of a closed circuit breathing apparatus. The adapter includes an exhalation valve disposed on the housing.

The exhalation valve can include a cap having at least one opening, a movable valve disc, and a spring element positioned between the valve disc and the cap. The spring element can be configured to bias the valve disc into a closed position during inhalation to maintain a positive pressure within the mask and configured to compress during exhalation allowing exhaled breath from the internal passageway to pass through the at least one opening in the cap. The second connecting end portion can include at least a first recess on at least a portion of an outer surface of the second connecting end portion. The recess can be configured to pass over or be captured by a corresponding feature in the mask providing a coupling between the device and the mask. The second connecting end portion can further include a release element that releases the coupling. The second connecting end portion can include a sealing element positioned around an outer surface of the housing. The first connecting end portion can include a valve element positioned within a portion of the first opening. The valve element can be configured to allow the passage of air during inhalation through the first opening into the internal passageway and prevent the passage of exhaled breath during exhalation from the internal passageway through the first opening. The valve element can be a flutter valve, flapper valve, check valve, ball check valve, clack valve, non-return valve, one-way valve, or a diaphragm check valve. The first connecting end portion can include a threaded inner surface or a plug-in connector assembly. The lung demand valve can be a second stage regulator of an open circuit breathing apparatus. The open circuit breathing apparatus can be a self-contained breathing apparatus.

In an interrelated aspect, disclosed is a system for adapting a mask of a closed circuit breathing apparatus for use with an open circuit breathing apparatus. The system includes a mask having a port configured to be reversibly connected to a closed circuit breathing apparatus. The system includes an adapter having a housing defining an internal passageway, a first connecting end portion of the housing having a first opening into the internal passageway, a second connecting end portion of the housing having a second opening into the internal passageway, and an exhalation valve disposed on the housing.

The first connecting end portion can be configured to mechanically and fluidically couple with a lung demand valve of an open circuit breathing apparatus. The second connecting end portion can be configured to mechanically and fluidically couple with the port of the mask. The second connecting end portion can include at least a first recess on at least a portion of an outer surface of the second connecting end portion. The at least a first recess can be configured to pass over or be captured by a corresponding feature in the port providing a coupling between the adapter and the mask. The second connecting end portion can further include a release element that releases the coupling. The second connecting end portion can include a sealing element positioned around an outer surface of the housing. The first connecting end portion can include a valve element positioned within a portion of the opening. The valve element can be configured to allow the passage of air during inhalation through the first opening into the internal passageway and prevent the passage of exhaled breath during exhalation from the internal passageway through the first opening. The valve element can be a flutter valve, flapper valve, check valve, ball check valve, clack valve, non-return valve, one-way valve, or a diaphragm check valve. The first connecting end portion can include a threaded inner surface or a plug-in connector assembly. The exhalation valve can be spring-biased into a closed position. The exhalation valve can include a cap having at least one opening, a movable valve disc, and a spring element positioned between the valve disc and the cap. The spring element can be configured to bias the valve disc into a closed position during inhalation to maintain a positive pressure within the mask and configured to compress during exhalation allowing exhaled breath from the internal passageway to pass through the at least one opening in the cap.

In an interrelated aspect, disclosed is an apparatus including a housing defining an internal passageway. The apparatus includes a first connecting end portion of the housing having a first opening into the internal passageway. The first connecting end portion is configured to mechanically and fluidically couple with a lung demand valve. The apparatus includes a second connecting end portion of the housing having a second opening into the internal passageway. The second connecting end portion is configured to mechanically and fluidically couple with a mask of a closed circuit breathing apparatus. The apparatus includes an exhalation valve disposed on the housing configured to maintain a positive pressure within the mask during inhalation and configured to allow exhaled breath from the internal passageway to pass therethrough during exhalation.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1A is a perspective view of an implementation of a CCBA system;

FIG. 1B is a view of the CCBA system with a portion of the protective housing removed;

FIG. 2A is a perspective backside view of a mask for use with the CCBA system;

FIG. 2B is a perspective, front side view of a mask for use with the CCBA system;

FIG. 3A is a perspective view of a port in the mask of FIG. 2B;

FIG. 3B is a perspective view of a breathing connector of a CCBA system;

FIGS. 4A and 4B are perspective views of an implementation of an adapter;

FIGS. 5A and 5B are views of the adapter of FIGS. 4A and 4B;

FIG. 6 is a cross-sectional view of the adapter of FIGS. 4A and 4B;

FIG. 7 is a perspective view of another implementation of an adapter.

Like reference symbols in the various drawings indicate like elements.

### DETAILED DESCRIPTION

Disclosed herein are devices, systems and methods to quickly, reliably and safely exchange between disparate breathing apparatuses, such as from a closed system to an open system (e.g. CCBA system to a SCBA system) without requiring the doffing and donning of a user's breathing mask.

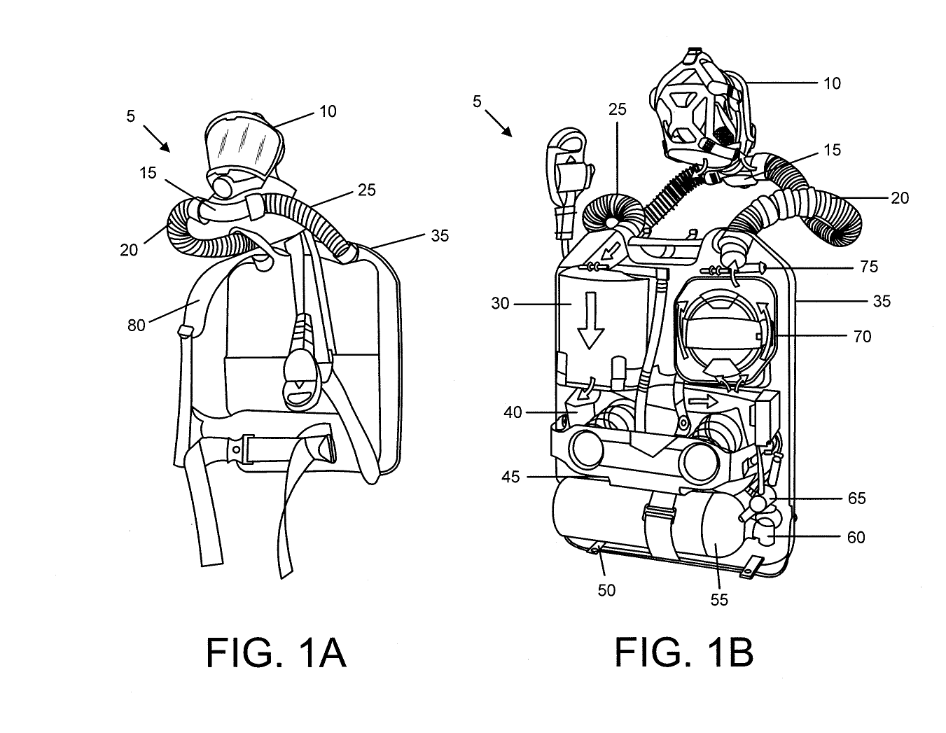

FIGS. 1A-1B show an implementation of a CCBA system 5. The CCBA system 5 can include the Drager PSS BG 4 PLUS (Drager Safety AG & Co. KGaA, Lubeck, Germany). The system 5 can have application in fire, military and law enforcement use as well as in industrial uses. The system 5 can be used in confined space applications such as buildings, mine shafts, tunnels or tanks, but it should be appreciated that the system 5 may be used in any environment in which there is a risk of exposure to dangerous substances in the surrounding atmosphere or reduced oxygen content in IDLH atmospheres.

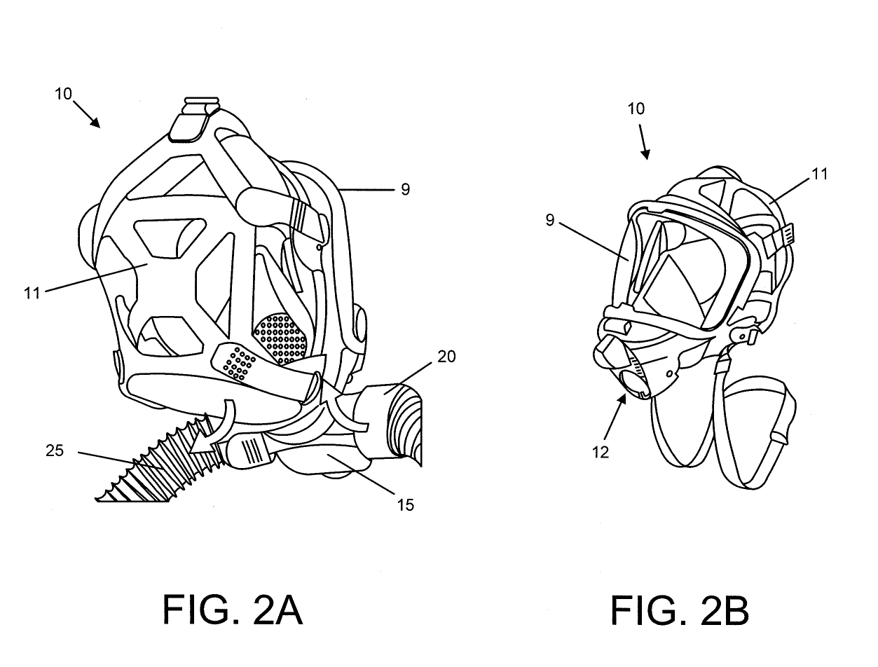

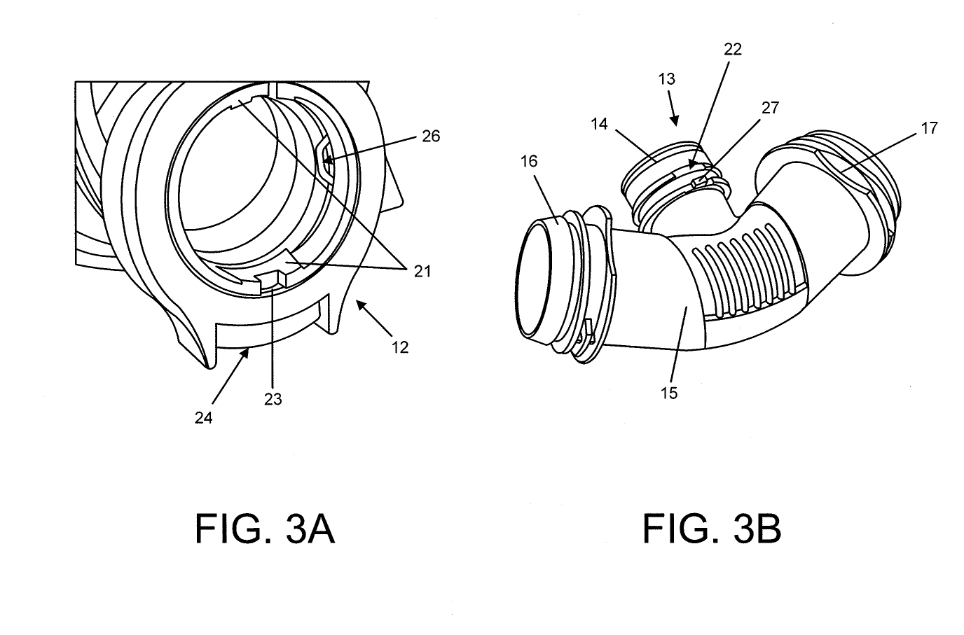

FIGS. 2A-2B show an implementation of a mask 10 that can be worn and used with the CCBA system 5. The mask 10 can include a face shield 9 for visibility and a head harness 11 for a secure fit. It should be appreciated that the mask 10 can include a fitted face piece, full face mask and/or hood type coupling. The mask 10 can include a port 12 for coupling with a corresponding port 13 in a connection duct 14 of a breathing connector 15 (see FIG. 3B).

As best shown in FIGS. 3A and 3B, the breathing connector 15 can have a first hose connection duct 16 for coupling with an inhalation hose 20 of the CCBA system 5 and a second hose connection duct 17 for coupling with an exhalation hose 25 of the CCBA system 5 such that the inhalation hose 20 and the exhalation hose 25 are fluidly communicating with the inside of the mask 10 for the user to breathe. The three ducts 14, 16, and 17 of the breathing connector 15 can form a t-shape or y-shape. The breathing connector 15 can include valving within the ducts. The valving can ensure the flow of breathable gas from the inhalation hose 20 is directed in a single direction towards the mask 10 through the first hose connection duct 16 and the flow of exhaled gas from the mask 10 is directed in a single direction away from the mask 10 through the second hose connection duct 17.

The connection duct 14 of the breathing connector 15 can insert through the port 12 of the mask 10. The connection duct 14 can have an o-ring or other sealing element around its outer surface such that the outer surface of the connection duct 14 seals with an inner surface of the port 12. As shown in FIGS. 3A and 3B, the inner surface of the port 12 of the mask 10 can include one or more features that are configured to interface with corresponding features on the connection duct 14 for proper connection between the breathing connector 15 and the mask 10. For example, the inner surface of the port 12 can include one or more raised features 21 and a recess 23 that are configured to interface with corresponding features on the outer surface of the connection duct 14. For example, the outer surface of the connection duct 14 can have one or more recesses 22 that can pass over the one or more raised features 21 and a post 27 that can insert within recess 23. The one or more raised features 21 on the inner surface of the port 12 can also prevent insertion of other components such as a lung demand valve to prevent inappropriate couplings with the CCBA. It should be appreciated that other types of connections between the connection duct 14 and the port 12 of the mask 10 are considered herein.

Again with respect to FIGS. 1A-1B, exhaled gas can be directed through the exhalation hose 25 towards a carbon dioxide absorber cartridge 30 contained within a protective housing 35 of the CCBS system 5. The protective housing 35 can also contain a breathing bag 40, a pressure relief valve 45, a drain valve 50, an oxygen cylinder 55, a cylinder valve 60, a pressure reducer 65, a breathing air cooler 70, and dosage connection 75. The dosage connection 75 can enrich the breathing gas with, for example at least 1.7 l/min oxygen and direct the gas into the inhalation hose 20 towards the breathing connector 15 coupled to the mask 10 for the wearer to inhale. The protective casing 35 can be configured to be worn or held such as with a plurality of straps 80.

The CCBA system 5 can have a limited time within which it can be used, such as for example 3-4 hours. After this time, the user can leave the toxic environment or in an emergency situation switch to another system. It can be desirable to replace the spent CCBA system or rescue a CCBA user having a malfunctioned CCBA system with an open, rechargeable system that provides respirable air such as a SCBA system or a Rapid Intervention Team (RIT) Pack. Because the CCBA system is a closed system, masks used with CCBA system do not include a second stage regulator or lung demand valve (LDV) or an exhalation valve, a valve that allows a wearer to push exhaled breath out of the mask into the ambient environment without letting ambient air from the environment back into the system. Whereas both LDV and exhalation valve are typically integrated within the mask of a SCBA system. However, removing the mask 10 of the CCBA system 5 to don the mask of a SCBA system takes extra time and puts the user at increased risk for exposure to the toxic environment during that time-period. The longer the user is exposed to the hazardous environment (and without respiratory protection) the greater the risk for the user to inadvertently or necessarily take a breath before the exchange is complete.

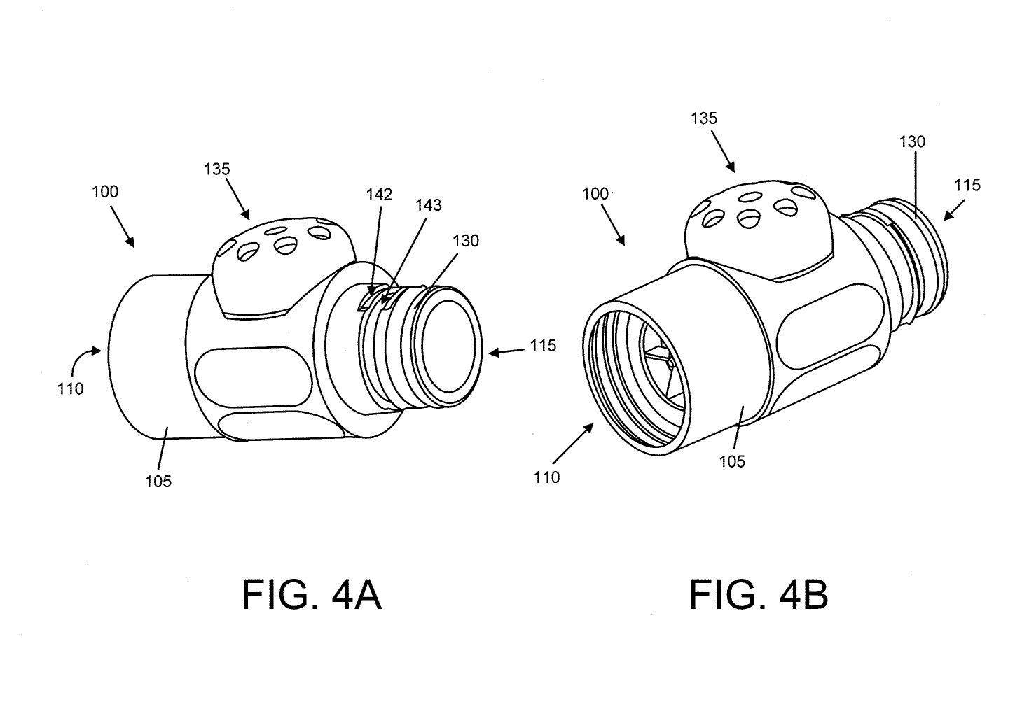

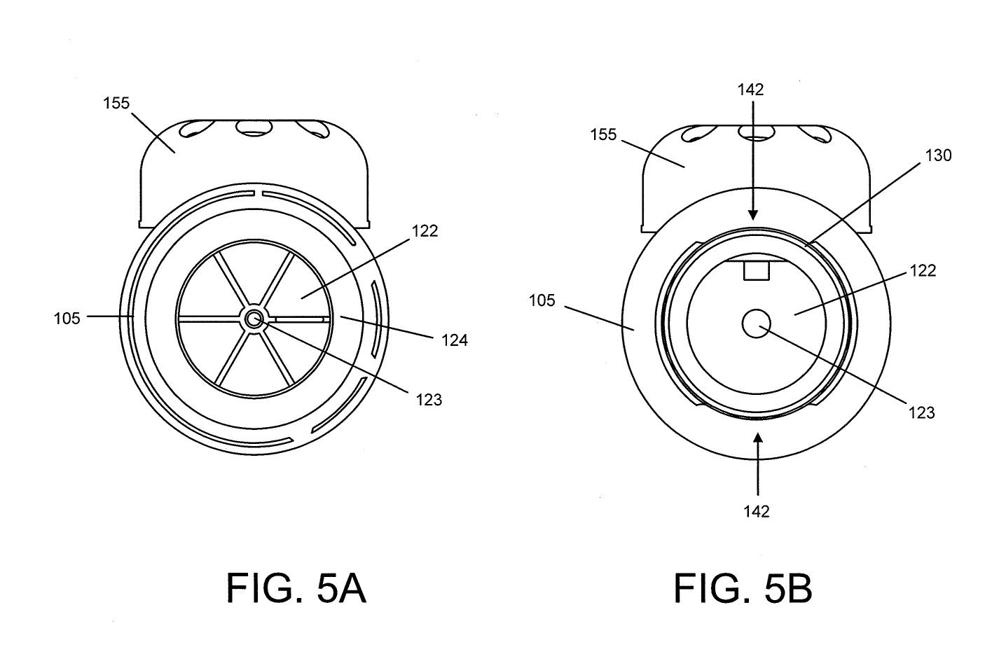

An implementation of an adapter 100 for use with the CCBA system 5 is illustrated in FIGS. 4A and 4B, FIGS. 5A and 5B, and FIG. 6. The adapter 100 allows for quick interchange between a closed system, such as the CCBA system 5, to an open circuit system, such as a SCBA system or a RIT Pack. Further, the adapter 100 allows a user to rapidly connect to the compressed air source without requiring the user to remove or change masks, which could potentially expose the user to environmental hazards. It should be appreciated that the adapter 100 can allow for an equally quick interchange from an open back to a closed system.

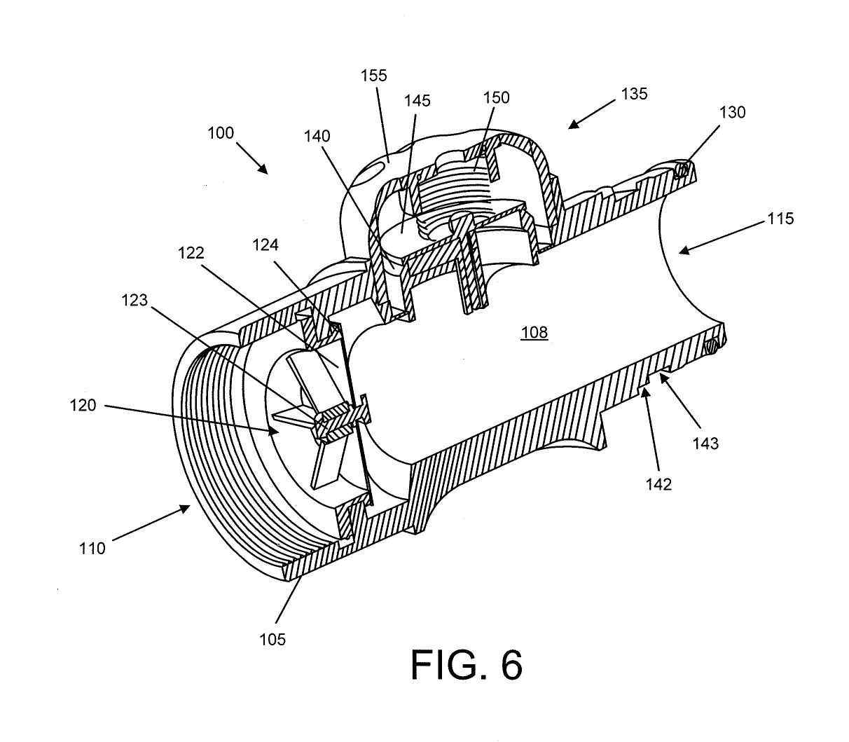

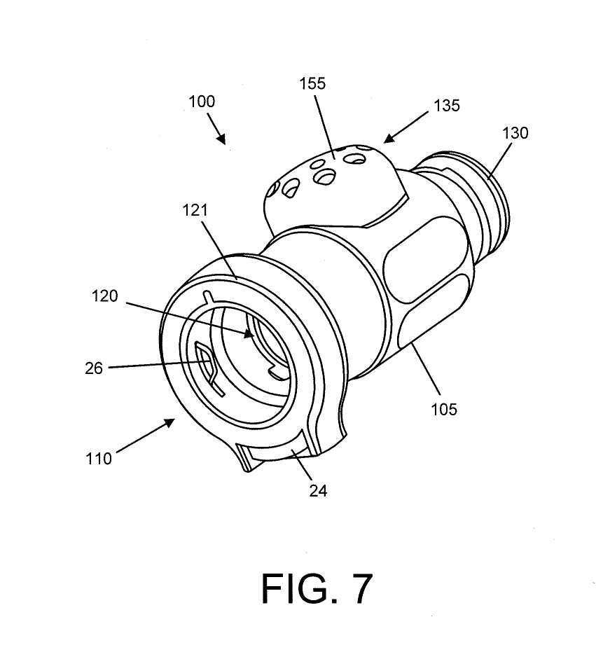

As best shown in FIG. 6, the adapter 100 can include a housing 105 having an internal passageway 108 extending between a first connecting end portion 110 and a second connecting end portion 115. The first connecting end portion 110 can be configured to connect with or accept therein an LDV (not shown). The configuration of the coupling between the first connecting end portion 110 and the LDV can vary. For example, in some implementations the inner surface of the first connecting end portion 110 can have threads (see FIG. 4B) having a pitch configured for threading with corresponding threads on an outer surface of a connecting end of the LDV. In another implementation, the first connecting end portion 110 can include a plug-in connector assembly 121 configured to accept the LDV (see FIG. 7). The inner surface of the plug-in connector assembly 121 can include one or more features configured to interface with corresponding features on the outer surface of the connecting end of the LDV providing proper connection between the LDV and the adapter 100. For example, an inner region of the plug-in connector assembly 121 can include one or more features configured to mechanically couple with corresponding features on the outer surface of the LDV connecting end. For example, one or more spring clips 26 can be positioned within the assembly 121. The spring clips 26 can be urged out of the way while the LDV passes through the assembly 121 and into end 110. The spring clips 26 can then snap into place within a corresponding recess in at least a portion of the outer surface of the connecting end of the LDV. To release such mechanical coupling between the adapter 100 and the LDV, a release element such as a button 24 can be depressed to remove the spring clip(s) 26 from the recess allowing for the LDV to be removed from the end 110 of the adapter 100.

The second connecting end portion 115 can be configured to connect with or insert into the port 12 of the mask 10 in place of the breathing connector 15. The second connecting end portion 115 can connect with the port 12 by interference fit and/or using corresponding mechanical elements that are keyed to the inner surface of the port 12 that help to prevent inadvertent removal of the adapter 100 from the port 12. As described above and as shown in FIG. 3A, the inner surface of the port 12 of the mask 10 can include one or more raised features 21. The outer surface of the second connecting end portion 115 can have one or more corresponding recessed features that mate with the one or more raised features 21 on the inner surface of the port 12. For example, the outer surface of the second connecting end portion 115 can have at least a first recess 142 that can pass over the one or more raised features 21 in the port 12. In one implementation, the inner surface of the port 12 can include two raised features 21 positioned opposite one another and the second connecting end portion 115 can have two corresponding recesses 142 that pass over the two raised features 21 in the port 12 allowing for a connection between the adapter 100 and the port 12. The raised features 21 and recesses 142 can be positioned such that the second connecting end portion 115 inserts within the port 12 according to a specific configuration relative to the mask 10. Further, the adapter 100 by virtue of the mating between the corresponding recesses 142 and raised features 21 can be prevented from rotating relative to the port 12 of the mask 10. This allows for the adapter 100 to be inserted in a pre-determined, specific orientation relative to the port 12 of the mask 10. It should be appreciated that the mating between the adapter 100 and the port 12 can vary. Further, the one or more corresponding features of the adapter and the port can have a variety of geometries and couplings.

The second connecting end portion 115 of the adapter 100 can include machined recess 143 on at least a portion of the outer surface of the second connecting end portion 115. Recess 143 can mechanically couple with another feature within the port 12, such as a spring clip 26 (see FIG. 3A). Upon insertion of the second connecting end portion 115 to the port 12, the spring clip 26 can be urged out of the way while the housing passes through the port 12 before the spring clip 26 snaps into place within recess 143. To release the mechanical connection between the adapter 100 and the port 12, one or both of the mask 10 and the adapter 100 can include a release element such as a button 24 that can be depressed to remove the spring clip 26 from the second recess 143 allowing for the second connecting end portion 115 of the adapter 100 to be pulled free from the port 12. The second connecting end portion 115 can have one or more sealing elements 130, such as an o-ring, positioned around an outer surface of the housing 105 to create a seal with the inner surface of the port 12 upon connection with the mask 10.

The first connecting end portion 110 can include a valve element 120 positioned therein that readily allows the flow of gas into the internal passageway 108 and prevents flow in the opposite direction. The valve element 120 can be a flap valve having a flexible disc 122, a pin 123 and a valve seat 124. The flexible disc 122 can be configured to move away from the valve seat 124. It should be appreciated that the configuration of the valve element 120 can vary, including but not limited to a variety of one-way valves including a flutter valve, flapper valve, check valve, ball check valve, clack valve, non-return valve, one-way valve, diaphragm check valve, or other valve through which fluid can flow in a single direction. As mentioned above, the first connecting end portion 110 can be coupled with a LDV. When connected to the LDV, inhalation by the user can result in air from a compressed air cylinder in the SCBA system moving into the first connecting end portion 110 through the valve element 120 causing the flexible disc 122 to move a distance in a direction away from the valve seat 124 such that the air flows into the internal passageway 108. Air in the internal passageway 108 can move through the internal passageway 108 towards the second connecting end portion 115, which when inserted through the port 12 of the mask 10 can be inhaled by the user.

The adapter 100 can also include an exhalation valve 135 disposed on the housing 105 that allows exhaled air to pass out from the internal passageway 108 of the adapter 100. The exhalation valve 135 can be spring-loaded to maintain a positive pressure in the user's mask 10 while breathing. The exhalation valve 135 can include a valve seat 140, a valve disc 145, a spring element 150 and a cap 155 having one or more openings. The spring element can be positioned between the valve disc 145 and the cap 155. The spring element 150 can bias the exhalation valve 135 into a closed position by urging the valve disc 145 against the valve seat 140 during inhalation. The spring element 150 is configured to allow the exhalation valve 135 to readily open upon exhalation. As the user exhales, the pressure of the exhaled breath urges the disc 145 of the exhalation valve 135 to travel a distance upwards away from the valve seat 140 compressing the spring element 150 allowing flow of the exhaled breath past the valve seat 140 and through the one or more openings within the cap 155 of the exhalation valve 135. The valve type of the exhalation valve 135 can vary, including but not limited to a variety of one-way valves including a flutter valve, flapper valve, check valve, ball check valve, clack valve, non-return valve, one-way valve, diaphragm check valve, or other valve through which fluid can flow in a single direction.

It should be appreciated that the relative configuration of the connecting end portions 110, 115 and the exhalation valve 135 can vary. In some implementations, the adapter 100 is in the shape of a T in which connecting end portions 110, 115 are opposite one another and exhalation valve 135 extends from a center region of the housing 105. The adapter 100 can also be in the shape of a Y or an L. Also, the exhalation valve 135 can be positioned such that it is aligned with the longitudinal axis of the housing 105 or orthogonal to the longitudinal axis of the housing 105. Further, the exhalation valve 135 can be positioned around the outer surface of the housing 105 in a variety of orientations relative to one or both of the connecting end portions 110, 115. For example, if the second connecting end portion 115 has a first recess 142 at 12:00, the exhalation valve 135 can be positioned on the housing at 12:00 or 3:00 or 6:00 or 9:00 or a variety of other positions in between. As mentioned above, one or both of the connecting end portions 110, 115 can be keyed such that the user is prevented from installing the adapter 100 in an orientation other than the proper orientation. Further, the adapter 100 can be keyed such that it is prevented from being installed into an improper system. For example, the inner surface of the first connecting end portion 110 can have threads into which only a LDV can be thread, such as due to the pitch of the threads. The plug-in assembly 121 used with some implementations of the adapter 100 can be configured to selectively connect with a LDV. Further, the second connecting end portion 115 can have one or more features (such as recess 142) on the outer surface that allows the adapter 100 to be inserted into the port 12 of a mask 10 appropriate for use with the closed system (CCBA), but may prevent the adapter 100 from being inserted into a port in a mask used with an open system (SCBA). One or both of the connecting end portions 110, 115 can be covered by a dust cap (not shown) when not in use or include a protective covering that can be pierced through, such as by the lung demand valve.

In use, the first connecting end portion 110 of the adapter 100 can be coupled to a LDV for use with a SCBA system prior to removing the breathing connector 15 from the port 12 of the mask 10 and installing the second connecting end portion 115 of the adapter 100 into the port 12. Alternatively, the breathing connector 15 can be removed and the second connecting end portion 115 of the adapter 100 can be installed into the port 12 prior to coupling the first connecting end portion 110 of the adapter 100 to the LDV for use with a SCBA system. The breathing connector 15 can be removed and the adapter 100 installed within seconds, preventing unwanted exposure to the environment during the exchange. The SCBA system can automatically begin providing respirable gas to the user through the adapter 100, such as upon the first breath.

The implementations set forth in the foregoing description do not represent all implementations consistent with the subject matter described herein. Instead, they are merely some examples consistent with aspects related to the described subject matter. Wherever possible, the same reference numbers will be used throughout the drawings to refer to the same or like parts.

Although a few implementations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows and steps for use described herein do not require the particular order shown, or sequential order, to achieve desirable results. Other implementations can be within the scope of the claims.

What is claimed is:

1. An adapter device comprising:
a housing defining an internal passageway;
a first connecting end portion of the housing having a first opening to the internal passageway, the first connecting end portion configured to mechanically and fluidically couple with a lung demand valve;
a second connecting end portion of the housing having a second opening into the internal passageway, the second connecting end portion configured to mechanically and fluidically couple with a mask of a closed circuit breathing apparatus; and
an exhalation valve disposed on the housing.

2. The device of claim 1, wherein the exhalation valve comprises:
a cap having at least one opening;
a movable valve disc; and
a spring element positioned between the valve disc and the cap,
wherein the spring element is configured to bias the valve disc into a closed position during inhalation to maintain a positive pressure within the mask and configured to compress during exhalation allowing exhaled breath from the internal passageway to pass through the at least one opening in the cap.

3. The device of claim 1, wherein the second connecting end portion comprises at least a first recess on at least a portion of an outer surface of the second connecting end portion.

4. The device of claim 3, wherein the at least a first recess is configured to pass over a corresponding feature in the mask providing a coupling between the device and the mask.

5. The device of claim 3, wherein the at least a first recess is configured to be captured by a corresponding feature in the mask providing a coupling between the device and the mask.

6. The device of claim 5, wherein the second connecting end portion further comprises a release element that releases the coupling.

7. The device of claim 1, wherein the second connecting end portion comprises a sealing element positioned around an outer surface of the housing.

8. The device of claim 1, wherein the first connecting end portion comprises a valve element positioned within a portion of the first opening, the valve element configured to allow the passage of air during inhalation through the first opening into the internal passageway and prevent the passage of exhaled breath during exhalation from the internal passageway through the first opening.

9. The device of claim 8, wherein the valve element is selected from one of the group consisting of a flutter valve, flapper valve, check valve, ball check valve, clack valve, non-return valve, one-way valve, and a diaphragm check valve.

10. The device of claim 1, wherein the first connecting end portion comprises a threaded inner surface.

11. The device of claim 1, wherein the first connecting end portion comprises a plug-in connector assembly.

12. The device of claim 1, wherein the lung demand valve is a second stage regulator of an open circuit breathing apparatus.

13. The device of claim 12, wherein the open circuit breathing apparatus is a self-contained breathing apparatus.

14. A system for adapting a mask of a closed circuit breathing apparatus for use with an open circuit breathing apparatus, the system comprising:
a mask comprising a port configured to be reversibly connected to a closed circuit breathing apparatus; and
an adapter comprising:
a housing defining an internal passageway;
a first connecting end portion of the housing having a first opening into the internal passageway;
a second connecting end portion of the housing having a second opening into the internal passageway; and
an exhalation valve disposed on the housing.

15. The system of claim 14, wherein the first connecting end portion is configured to mechanically and fluidically couple with a lung demand valve of an open circuit breathing apparatus.

16. The system of claim 14, wherein the second connecting end portion is configured to mechanically and fluidically couple with the port of the mask.

17. The system of claim 14, wherein the second connecting end portion comprises at least a first recess on at least a portion of an outer surface of the second connecting end portion.

18. The system of claim 17, wherein the at least a first recess is configured to pass over a corresponding feature in the port providing a coupling between the adapter and the mask.

19. The system of claim 17, wherein the at least a first recess is configured to be captured by a corresponding feature in the port providing a coupling between the adapter and the mask.

20. The system of claim 19, wherein the second connecting end portion further comprises a release element that releases the coupling.

21. The system of claim 14, wherein the second connecting end portion comprises a sealing element positioned around an outer surface of the housing.

22. The system of claim 14, wherein the first connecting end portion comprises a valve element positioned within a portion of the opening, the valve element configured to allow the passage of air during inhalation through the first opening into the internal passageway and prevent the passage of exhaled breath during exhalation from the internal passageway through the first opening.

23. The system of claim 22, wherein the valve element is selected from one of the group consisting of a flutter valve, flapper valve, check valve, ball check valve, clack valve, non-return valve, one-way valve, and a diaphragm check valve.

24. The system of claim 14, wherein the first connecting end portion comprises a threaded inner surface.

25. The system of claim 14, wherein the first connecting end portion comprises a plug-in connector assembly.

26. The system of claim 14, wherein the exhalation valve is spring-biased into a closed position.

27. The system of claim 14, wherein the exhalation valve comprises:
a cap having at least one opening;
a movable valve disc; and
a spring element positioned between the valve disc and the cap,
wherein the spring element is configured to bias the valve disc into a closed position during inhalation to maintain a positive pressure within the mask and configured to compress during exhalation allowing exhaled breath from the internal passageway to pass through the at least one opening in the cap.

28. An apparatus comprising:
a housing defining an internal passageway;
a first connecting end portion of the housing having a first opening into the internal passageway, the first connecting end portion configured to mechanically and fluidically couple with a lung demand valve;
a second connecting end portion of the housing having a second opening into the internal passageway, the second connecting end portion configured to mechanically and fluidically couple with a mask of a closed circuit breathing apparatus; and
an exhalation valve disposed on the housing configured to maintain a positive pressure within the mask during inhalation and configured to allow exhaled breath from the internal passageway to pass therethrough during exhalation.
